(19) United States
(12) Patent Application Publication
(10) Pub. No.: US 2025/0216328 A1
(43) Pub. Date: Jul. 3, 2025

(54) WHITE LIGHT SCATTERING IN OPTICAL BIOMOLECULE INTERACTION ANALYSIS

(71) Applicant: Miltenyi Biotec B.V. & Co. KG, Bergisch Gladbach (DE)

(72) Inventor: Heinrich SPIECKER, Bergisch Gladbach (DE)

(73) Assignee: Miltenyi Biotec B.V. & Co. KG, Bergisch Gladbach (DE)

(21) Appl. No.: 18/849,683

(22) PCT Filed: Mar. 30, 2023
(86) PCT No.: PCT/EP2023/058332

§ 371 Date: Sep. 23, 2024

(30) Foreign Application Priority Data
Mar. 30, 2022 (DE) 10 2022 107 593.1

(51) Int. Cl. G01N 21/47; G01N 33/543

(52) U.S. Cl. CPC G01N 21/4788; G01N 33/54373; G01N 2201/0634

(57) ABSTRACT

The invention is directed to a device for the detection of target molecules, comprising

a transparent substrate provided with binding sites on one surface of the substrate, wherein the binding sites are capable of binding at least one target molecule a light source

means for coupling light provided by the light source into the substrate, wherein at least a part of the light generates an evanescent field of light propagating along the surface provided with the binding sites,

wherein the evanescent field of light is diffracted by target molecules bound to the binding sites, thereby creating at least one detection signal which is detected by at least one detector

characterized in that the light source provides low coherent or non-coherent light and the dispersion of the detection signal generated by the diffraction of low coherent or non-coherent light is reduced by at least 50% by one or more dispersive elements.

The invention is associated to the field of sensor instruments for optical and label-free detection of biomolecule interactions.

### BACKGROUND

Label-free detection of biomolecule interactions can be realized by a variety of methods, which can be classified according to their respective detection principle, e.g. calorimetric, electrochemical, acoustic and optical methods. Optical methods have in common that the association of biomolecules or analytes to their ligands or binders induces a change of light guided along or through the active sensor surface. For example, the intensity of light or its phase is altered due to biomolecule association and this change can be detected in real-time to determine binding kinetics and analyte concentration in solution (1).

The most widely used method in the field of label-free biomolecule interaction analysis (BIA) with optical transducer is based on a technology referred to as “Surface Plasmon resonance” (SPR). In this method, the change in the refractive index of light reflected from a metal surface is detected (refractometric sensor. Binding of biomolecules to the other side of this surface leads to a change in the refractive index, which is proportional to the mass added to the sensor surface. This method offers high sensitivity (appr. 10 pg/mL), but it is prone to changes in media conditions (media composition, temperature) and non-specific binding and requires a reference channel (2). SPR-based Biacore instruments, distributed by Cytiva Life Sciences, are leading the market in the field of optical BIA (https://www.cytivalifesciences.com/en/us/about-us/our-brands/biacore). These instruments can have a limit of detection (LOD) down to 0.01 ng/cm², depending on the molecular weight of analytes and binders.

Another method that is widely employed in the field is called Biolayer Interferometry (BLI). Here, the interference pattern of light reflected from two surfaces, a layer of immobilized protein and an internal reference layer, is analyzed. Specific interferometric wave patterns are shifts by changes in protein association on an optical fiber surface, which is dipped into the analyte solution (3). Main vendor of BLI instruments is Sartorius with its Octet system series (https://www.sartorius.com/en/products/protein-analysis/octet-label-free-detection-systems). Although these instruments have some advantages, as no fluidics are necessary and shaking of the microtiter plate keeps analyte solutions homogencous. But compared to other technologies in the field, the LOD of BLI with 0.1 ng/cm² is rather high and only analytes with a molecular weight higher than 150 Da can be detected. Another variant of interferometric BIA, the waveguide-based “Grating-Coupled Interferometry” (GCI) offers kinetic measurements with faster off-rates than classical BLI (k_{d}=10 s⁻¹ vs. 0.1 s⁻¹) and is commercialized by Creoptix (https://www.creoptix.com/technologies/gci). GCI was proven to detect analytes with a molecular mass of 59 Da at signals lower than 1 pg/mm² (4). A new method interferometric commercialized by Refeyn (https://www.refeyn.com/) is called “Mass Photometry”. The technology is based on “Interferometric scattering microscopy” (iSCAT, (5) and detects lights scattered from single analytes coming in close proximity to their respective binder. Compared to other systems, it can also detect smaller analytes down to 30 kDa.

Another group of optical BIA instruments utilize planar waveguides guiding coherent light to diffractive gratings. The associated technology named “Focal Molography” was originally published by ETH Zurich University in cooperation with Roche Diagnostics GmbH. Here, Molograms, nanopatterns of molecular recognition sites, create light diffraction. Specific binding of target molecules to the mologram increases light diffraction, forming a coherent signal. The technology offers many advantages, as measurements are not affected in crude samples, independent on refractive index of the analyte solution and temperature changes. The LOD of 0.01 ng/cm² is comparable to Biacore devices. As the sensitivity of this technology can be seen as a function of the molecular mass of binders and analytes, small analytes with a low molecular weight need to be introduced into the system in high concentration, limiting its usability e.g. in cytokine diagnostics from blood samples. Another hurdle is that not only binders have to be attached to specific regions of the Mologram, but the adjacent regions need to b backfilled with “non-binders” of equal molecular weight. This complicates the production of the required sensor chips. Furthermore, the invention disclosures filed in this subfield claim that the use of coherent light is unavoidable for the respective optical setups, increasing light source costs (WO2013107811, WO2014086789, WO2015004264, WO2015007674, WO2019166562, EP3428622B1, EP3428622B1, EP3835764A1).

The prior art related to “Label-free detection of biomolecules” is for example disclosed in the following publications:
(1) Nirschl M, Reuter F, Vörös J. Review of transducer principles for label-free biomolecular interaction analysis. Biosensors (Basel). 2011; 1(3): 70-92. Published 2011 Jul. 1. doi: 10.3390/bios 1030070.
(2) Nguyen H H, Park J, Kang S, Kim M. Surface plasmon resonance: a versatile technique for biosensor applications. Sensors (Basel). 2015; 15(5): 10481-10510. Published 2015 May 5. doi: 10.3390/s150510481.
(3) Concepcion J, Witte K, Wartchow C, et al. Label-free detection of biomolecular interactions using BioLayer interferometry for kinetic characterization. Comb Chem High Throughput Screen. 2009; 12(8): 791-800. doi: 10.2174/138620709789104915.
(4) Jankovics H, Kovacs B, Saftics A, et al. Grating-coupled interferometry reveals binding kinetics and affinities of Ni ions to genetically engineered protein layers. Sci Rep 10, 22253 (2020). doi: 10.1038/s41598-020-79226-w.
(5) Young G, Kukura P, Interferometric Scattering Microscopy. Ann Rev Phys Chem 70, 301-322 (2019). doi: 10.1146/annurev-physchem-050317-021247 Gatterdam, V, Frutiger, A, Stengele, K P, et al. Focal molography is a new method for the in situ analysis of molecular interactions in biological samples. Nature Nanotech 12, 1089-1095 (2017). Doi: 10.1038/nnano.2017.168.

### OBJECT OF THE INVENTION

The aim of the invention disclosed in this application is related to the detection of scattered light from an ensemble of binding molecules where the measured signal changes if analytes bind to a sub-ensemble of the ensemble of molecules. The invention discloses methods to reduce complexity and cost of not only the device but also consumables, increase sensitivity an broadens the possibility of application especially in situations, where the customer wishes to adapt the system to a specific need.

In the prior art, coherent light is needed to confine the signal and to increase the signal to noise ratio. The signal is enhanced since the scattered light from the individual molecules arranged in a well-defined pattern contributes coherently to one point at a detector which is typically a camera sensor. This also requires a spectral coherence of the excitation light in units of spectral bandwidth divided by the center wavelength approximately 1/1000. This results in a prominent speckle background at the detector. The origin of the speckles is either scattered light from molecules non-specifically bound to the surface and from excitation light scattered and reflected within the sensor chip or apparatus. One aspect of the invention is to reduce the need of spectrally coherent light and thus also reduces the speckles at the detectors as a source of errors. It also enables the use of cheaper light sources.

Accordingly, object of the invention is a device for the detection of target molecules, comprising
a transparent substrate provided with binding sites on one surface of the substrate, wherein the binding sites are capable of binding at least one target molecule
at least one light source
means for coupling light provided by the light source into the substrate, wherein at least a part of the light generates an evanescent field of light propagating along the surface provided with the binding sites,
   wherein the evanescent field of light is diffracted by target molecules bound to the binding sites, thereby creating at least one detection signal which is detected by at least one detector characterized in that the light source provides low coherent or non-coherent light and the dispersion of the detection signal generated by the diffraction of low coherent or non-coherent light is reduced by at least 50%, preferable by at least 80%, more preferable by at least 95% and most preferred by at least 99% by one or more dispersive elements.

The reduction of dispersion is calculated by the ratio of dispersion obtained with and without using dispersive elements. In absolute values, the dispersion should be less than 10 mikrometer/nanometer wavelength of the light.

The dispersive elements can be located in the path of light before and/or after the light is diffracted by the target molecules bound to the binding sites.

In another aspect of the invention the wavelength is repeatedly swapped in its center wavelength and a set of data is acquired for various wavelengths of the source. This further reduces noise of the signal by combining the data.

In another aspect of the invention spatio-temporal modulation of the light is used to eliminate the speckles generated at the detector. A further aspect of the invention is an increase of sensitivity by a more distinct immobilization pattern and the readout of more than one diffraction order of the diffracted light.

The following description of the drawings disclose the main aspects of the invention. The aspects shown can be combined by persons skilled in the art in various ways. Thus the invention covers also all combination of the disclosed aspects.

### BRIEF DESCRIPTION OF THE DRAWINGS

FIG. 1: Device for label-free biomolecule interaction analysis. A: Diffractive sensor with different binding zones (04) within a continuous pattern of straight lines (03) on a sensor chip with planar waveguide (02) with microlens array (09) for photodetection (11). Upper panel: Top view of the waveguide (02). Lower panel: Side view of the waveguide (02) with optical detection pathway. B: Alternative setup according to A: Lines (03) are only generated inside the binding zones (04). C: Variants for microlens array (09): Upper panel with 3 microlenses for formation of 3 foci, middle panel with 5 microlenses for formation of 5 foci, lower panel with one lens for formation of a single focus.

FIG. 2: Diffractive sensor generating individual diffracted planar wavefronts (03) restricted to binding zones (04). The waveguide (02) directs the light (01) to a hologram with lens-phase pattern containing binding structures (03) positioned with increasing proximity to each other, causing a different diffraction pattern for each binding zone (04), guiding the light (01) diffracted from different binding zones (04) to a common center, but not bundling it. A telecentric lens (12) generates single foci (10) on the photodetector (11).

FIG. 3: A diffractive sensor as described in FIG. 2, bundling the diffracted light (01) from different binding zones (04).

FIG. 4: Diffractive sensor with non-coherent light source. Light (01) from a white light source (14) is filtered (15) to become monochromatic red light. Monochromatic light (01) is reflected by a binding zone-shaped grating (13), creating a dispersed first or higher order diffracted beam. A minor portion of light (01) is not reflected and serves as internal reference. The major portion of light (01) is reflected again by the rear surface (16) of the sensor chip to guide it to the binding zones (04) inside the flow cell (18) and create a signal in a light-covered detection space (19) subsequently. Excess light (dotted lines) is led out of the system into beam dumps (17).

FIG. 5: A simplified version of a diffractive sensor as described in FIG. 4 with direct light (01) guiding from grating (13) to binding zone (04).

FIG. 6: Diffractive sensor with non-coherent light source and 3D volume holographic grating for filter integration. Monochromatic light (01) is guided into a volume hologram (20), creating dispersion and redirecting it to the binding zones (04) inside the flow cell (18). Focal spots (10) are created in a light-covered detection space (19) subsequently. A second volume hologram (20) assures scattering and can be used as reference.

FIG. 7: Diffractive sensor with coherent or non-coherent light source and grating outside of the sensor chip (02). Light (01) is guided by a mirror (16) onto a grating (13), creating dispersion and reflecting it to the binding zones (04) inside the flow cell (18).

FIG. 8: Diffractive sensor with grating and binding zones positioned in row on the sensor chip with planar waveguide. Light (01) is directed from a first grating by a planar waveguide (black arrow) to the binding zones (04). A: diffracted light from the binding zones (04) is focused by two telecentric lenses (12) between an aperture (06) before it reaches a grating (13), guiding it to the photodetector (11). B: Light (01) form binding zones (04) is directly guided to the grating (13).

FIG. 9: Diffractive sensor with grating downstream of binding zones on a sensor chip. Light (01) is guided to the binding zones (04) on the upper side of the sensor chip (02). Diffracted light (01) is then directed to a grating (13) on the lower side of the chip (02, parallel to the binding zone), diffracting it towards a lens (07) that focusses the signal on a photodetector (11).

FIG. 10: Diffractive sensor with reference gratings. A: Reference gratings (13′) are positioned in front and behind the binding zones (04) on a sensor chip (02). Diffracted light (01) is bundled by a lens (07), creating a specific pattern on the detector (11), depending on the position of binder and analyte (25) association on or next to the lines with binding structures (03). C: A volume hologram (20) is used for reference generation.

FIG. 11: Diffractive sensor with immersion layer. A grating (13) is optically coupled to the chip (02) with an immersion fluid (23). The grating (13) is positioned in a plane parallel to the plane of the binding zone (04).

FIG. 12: A method for binder modulation on a sensor chip for binding zone definition. A: Binders (23) are coupled to the surface of the sensor chip (02) on predefined lines with binding structures (03). B: Along a planar waveguide, coherent light (01) from a pulsed laser deactivates binders (24′) in a specific pattern according to wave propagation. C: During the experiment, analytes (25) can only attach to non-deactivated binders (23).

FIG. 13: Diffractive sensor with dual function. With the same device, binding sites and non-binding sites (04) can be printed on chips (02) by stepwise spatial illumination and subsequently detection of analyte binding. Two separate optical paths are used for excitation light and for printing (01′) and for detection (01). A beam splitter (21) separates the detection light (01) into two beams and guides them into the sensor chip (02), passing a dispersive element, i.e. a grating (13).

FIG. 14: Diffractive sensor with dual function. With the same device, binding sites and non-binding sites (04) can be printed on chips (02) by stepwise spatial illumination and subsequently detection of analyte binding. The same optical path is used for excitation light and for printing (01′) and for detection (01). Both printing light (01′) beams are directed through a layer of immersion liquid (23) towards the chip (02) and interfere at the surface. This directly generates the binding zone (04) necessary to bind the analytes. By shifting the beam splitter (21) or tilting one of the directing mirrors (16) the phase of the interference at the sample can be changed.

FIG. 15: Variant of the diffractive sensor with dual function depicted in FIG. 13. The light beam (01′) for the activation of the chip is modulated by means of an DLP device (22). The modulated beam is than split in two beams (21) and is than redirected to the chip (02). In this example a coupling grating (13) on the chip is used to couple the two beams into the chip and direct them to the surface where the grating is activated, forming a binding zone (04). In this example the same grating can also be used to couple the detection light beam (01) into the chip.

FIG. 16: Workflow for diffractive sensor with dual function that can print binding sites and non-binding sites on chips by stepwise spatial illumination and subsequently detect binding of analytes. A: In step a, the binding zone area of the chip is covered with photoprotective groups before illumination of distinct areas of the binding zones with UV light to cleave photoprotective groups and attach binders (b). In step c, the diffraction signal increase is measured with red light until binder coverage is sufficient. After a phase shift, remaining areas are illuminated with UV light to cleave photoprotective groups and attach non-binders (d). During this step, the diffraction signal decrease is measured with red light until binder signal is completely extinct (c). Optionally, random phase UV illumination is applied to block remaining binding sites (f). Finally, the addition of an analyte to determine concentration and binding kinetics by illumination with red light (g). B: A graph showing the process described in A, signal intensity is described as a function of time. Phase shift and random phase illumination are marked by the dotted lines. The Workflow may also includes elongated steps to saturate the activation which causes a higher confinement of the generated structure and generates contributions to multiples of the fundamental spatial frequency.

FIG. 17: Variants of the diffractive sensor for strong reduction of speckles applicable for coherent or non-coherent light sources by spatio-temporal modulation (SLM). Light (01) guided through a variable mask or SLM (26 in A and C) is illuminating the surface of the chip (02) or after spatio-temporal filtering (26 in B), light is emitted from the surface of the chip (02) and collected at the detector (11). In C, a scanner or MEMS-scanner (27) can to direct the light to the subareas.

FIG. 18: Variant of the diffractive sensor for strong reduction of speckles applicable for coherent or non-coherent light sources by spatio-temporal modulation (SLM) FIG. 17) combined with a chip (02) where the light (01) is coupled to a waveguide.

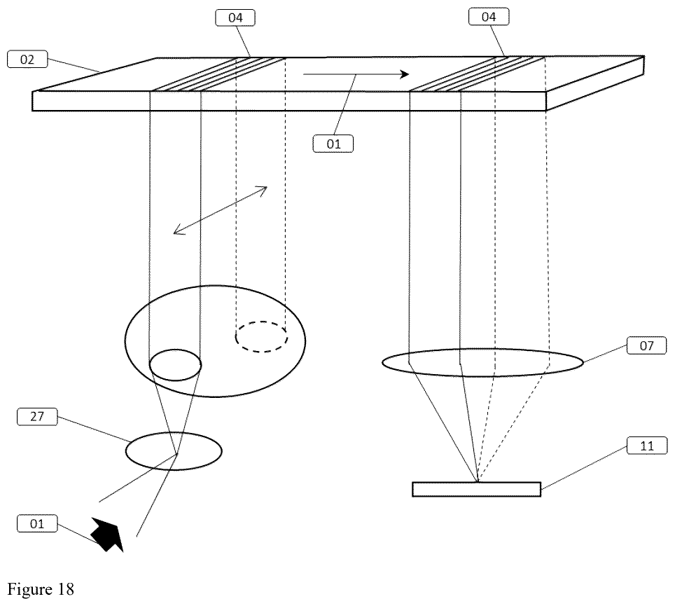

### GLOSSARY

### Structures & symbols

01 Light for binding detection
01′ Light for chip surface activation
02 Transparent substrate
03 Line with binding structures
04 Binding zone
Beam path
06 Aperture plate
07 Lens or objective
08 Pinhole aperture
09 Microlens array
Focus
11 Detector
12 Telecentric lens
13 Grating
13′ Reference grating
13′ Spatial light modulator grating
14 Light source for binding detection
14′ Light source for chip surface modulation
Filter
16 Mirror or reflective surface
17 Beam dump
18 Flow cell
19 Light-protected room
Volume-holographic grating
21 Beam splitter
22 Digital light processing (DLP) projector
23 Glass slide with immersion liquid
24 Binder
24′ Deactivated binder
Analyte
26 Variable mask or spatial light modulator
27 Scanner

### DESCRIPTION OF THE INVENTION

The light provided by the light source is low coherent or non-coherent light. Such light has preferable a spectral bandwidth wider than 1/100, more preferable wider than 1/1000 and most preferred wider than 1/10000 of the center wavelength of the light. Independent from the spectral bandwidth, the light may have a wavelength in the visible range (so called white light), i.e. between 250 and 1000 nm.

The device of the invention may comprise more than one light sources (like 2) which may provide light beams with the same or a different wavelength or range of wavelength.

In all embodiments described hereinafter, the dispersive elements may be gratings and/or coupling gratings and/or prisms and/or volumetric holograms and/or tilted interfaces. Further, holographic gratings, volume gratings spatial light modulators or DMDs can be used as dispersive elements.

In one embodiment of the invention, a plurality of binding sites is provided at different locations on the substrate, thereby generating a plurality of detection signals and wherein the detection signals are individually focused on the detector using at least one array of optical elements.

The arrays of optical elements may comprise lenses and/or microlenses and/or facetted elements and/or rotating apertures or sliding apertures. It is possible to use several arrays of optical elements, which can have the same or different optical properties. In a variant thereof, different arrays of optical elements with different optical properties are provided wherein the different arrays of optical elements are exchangeable. In another variant, the arrays of optical elements are provided with at least one aperture plate and/or an aperture plate array.

In another embodiment, the detection signals may be space-filtered by at least one optical element located in the Fourier plane of the plane of the binding sites.

The detection signal is focused by the space-filtering optical elements in an intermediate Fourier plane and finally focused and detected by at least one detector. The plane of the binding sites can be identical to the surface plane of the substrate.

The optical elements for space filtering may select at least one diffraction order of the detection signal.

The at least one optical element can be any optical means capable of space-filtering light, for example a diaphragm and/or a pinhole (8) as long as the elements are in or close to the Fourier plane of the plane of the binding sites.

The substrate may comprise a planar waveguide and/or a prism and/or independent thereof, at least one coupling surface for means for coupling a beam of light.

Optionally, the binding sites are arranged on one surface of the substrate in a plurality of lines having the same or different pitch. The substrate itself may be produced from glass or transparent polymers.

In a further embodiment, light provided by the light source is spatio-temporal modulated with a variable mask, a spatial light modulator or a scanner.

The device of the invention is directed to detecting/analyzing all sorts of target molecules, such as RNA, DNA, c-DNA, single stranded DNA, peptides, proteins and oligonucleotides. Accordingly, the binding sites should be designed to bind such target molecules as specific as possible. In general, the device is not restricted to any binding chemistry and a person skilled in the art is well aware of the binding chemistry which can be used for such purposes. For example, the binding sites provided on the surface of the substrate may be selected from the group of antibodies or fragments thereof of nucleic acids.

The substrate used in the device of the invention is not of particular importance as long as the light coupled into the substrate generates an evanescent field of light propagating along the surface provided with the binding sites. Suitable substrates are glass or transparent polymers. In the following, the terms “binding zones” and “subareas” are used interchangeable.

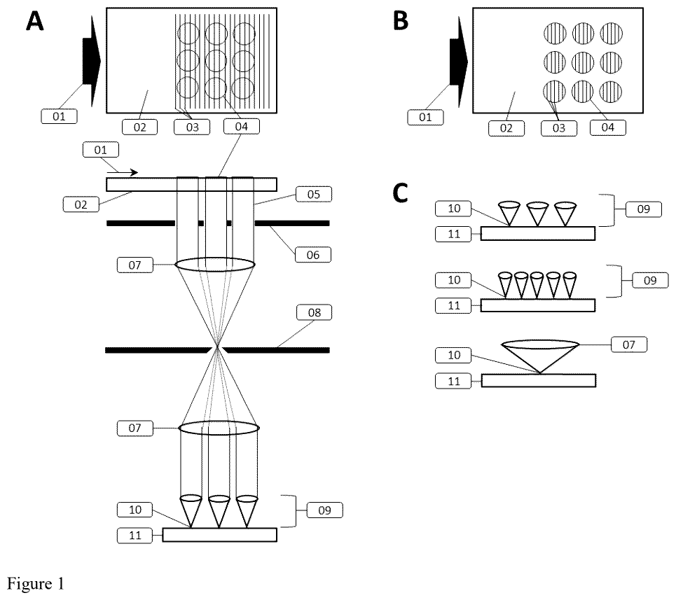

In variant of the invention, different binding zones (04) are defined at specific positions of a continuous pattern of straight lines with binding sites (03) on a sensor chip with planar waveguide (02) FIG. 1). Scattered coherent light (01) is optionally passed through an aperture plate (06) before it is focused by a lens (07). After passing a pinhole (08), light is collected by lens (07) and directed to a microlens array (09), generating single foci (10) on a detector (11). The microlenses focus the light scattered from the binding zones to the detector. Optionally, the lines are only generated inside the binding zones (04) to reduce background signal. The microlens array (09) might also consist of other pluralities of microlenses than depicted in the figure FIG. 1A). Alternatively, in this and all following embodiments, the microlens array (09) could be replaced by facetted elements or rotating or sliding apertures selecting the light from individual binding zones sequentially (not depicted). In an more elaborated embodiment the array of microlenses (09) can be changed to an microlens array with a higher or lower number of individual microlenses or just removed to adapt the device to different sensor chips with lower or higher numbers of binding zones FIG. 1B and C). The binding zones might be generated by spotting different binders to the binding zones or by optically activating a crosslink between the binders and the surface. The pattern of the binding zones has to be according to the microlens array (09) used in the detection beam path. The advantage of the embodiment is, that the structure of the sensor chip is simple. The generation of one planar wave enables simple remote detection, where the distance between the sensor and the detector unit can be large. This reduces the contribution of scattered light on the sensor. Different types of apertures (FIG. 1A, 06 and or 08) may be inserted to further reduce scattered light. One further advantage is that the simple structure of the sensor chip also enables an easy production of the chip since the pattern may be generated just by an interference of two planar beams of activating light (not depicted).

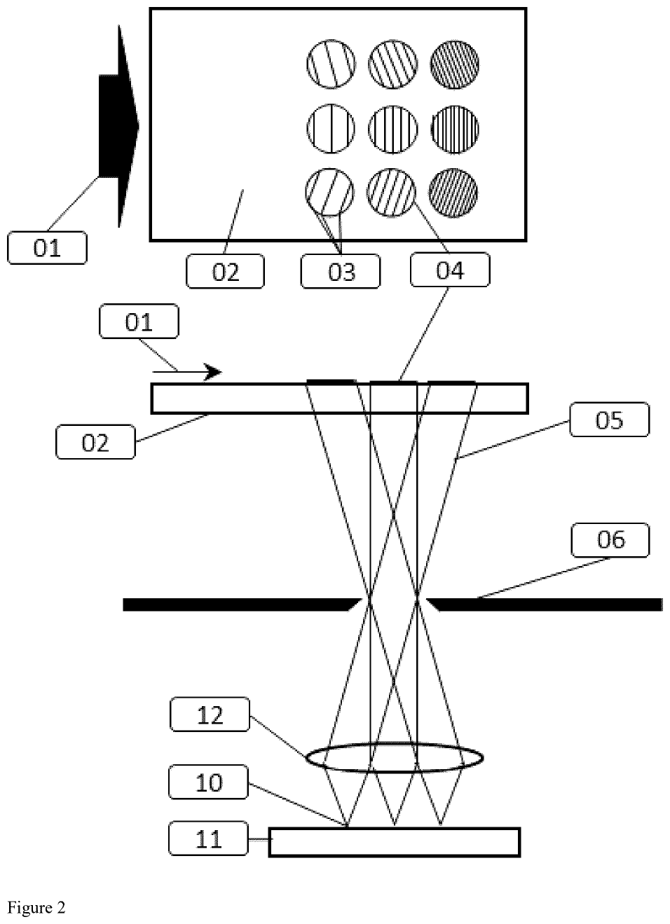

In another version of the previous embodiment, the diffractive sensor does not contain a continuous pattern of straight lines with binding structures (03), but an alternative grating structure with individual straight gratings generating individual diffracted planar wavefronts (03) generated by the binding zones (04) (FIG. 2). The angle and grating period is designed in a way that the signal light (01) scattered from the binding zones (04) entered a common aperture (06). After passing the aperture (06), light is directed to a lens ideally with a telecentric design (12), generating single foci (10) of the light from the binding zones (04) on the photodetector (11). In this embodiment the straight beamlets emitted from the individual binding zones can be directed to a far common aperture. This enables the reduction of scattered light.

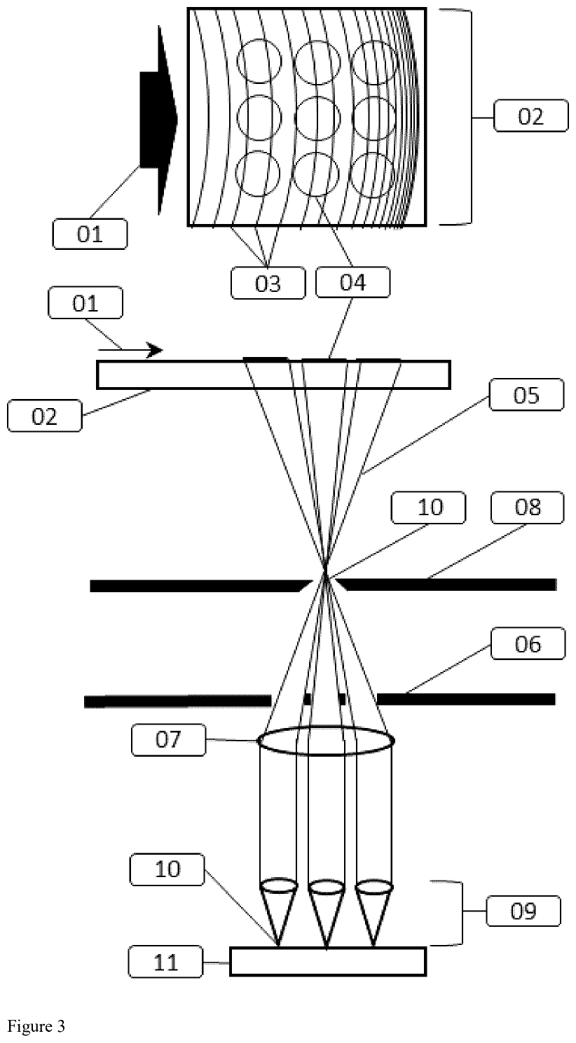

In further version of the second embodiment, the diffractive sensor with an alternative hologram with individual gratings (03) is coupled to another detection pathway (FIG. 3). The waveguide (02) directs the light (01) to a collimating pattern containing binding structures (03) generating a common spherical wave of diffracted light from all different binding zones (04) focused to a common first focus (10) within a pinhole (08) with acts as a global space filter. After passing the pinhole (08), the signal light is directed to a lens (07) ideally with telecentric design (12) and to a subsequent microlens array, generating single foci (10) that are focused onto a detector (11). The signal light (01) is optionally passed through an aperture plate (06) before or after it is focused by the lens (07) to remove background signal. The embodiment has the advantage that a real space filter eliminates most of the stray light. Therefore in this setup, the lines do not need to be restricted to the binding zones (04) to reduce background light. As described in one of the other embodiments the microlens array (09) could be exchangeable to adapt the device for different geometries of binding zones. The additional lens (07) is used to collimate the bundle of light and to hit the microlens array along its axis. The lens might also be eliminated at the cost of telecentricity on the detector (11). In this embodiment the production of the sensor chip can be done by an interference of one planar activating beam with one spherical activating beam resulting in the required segment of a Fresnel-zone type pattern (not depicted).

In further embodiments, specific optical setups allow to simplify the sensor chip. They also allow for the use of non-coherent light and include one or more gratings that can be for example, relief structures, holographic amplitude of phase gratings, volume-holographic gratings, i.e. a spatial light modulator that is used as holographic grating. The additional gratings are inserted in the excitation beam path directly or as part of the sensor chip or in the detection beam path directly or as part the sensor chip. The light sources used might be lasers, super luminescent diodes, broad band Vertical Emitting Surface Emitting Laser (VESCL), LEDs, spectral lamps or white light lasers e.g. generated in a photonic crystal fiber. In all embodiments where grating structures are used to extend the spectral range of the light which can be transmitted and focused on the detector it is also possible to use a tunable or spectrally switchable light source. Then a series of data for different wavelength can be acquired and accumulated to further increase the signal to background ratio. The advantage of a serial readout for different wavelengths is that remaining dispersion leading to different positions of the signals for different wavelengths can be corrected by numerical methods.

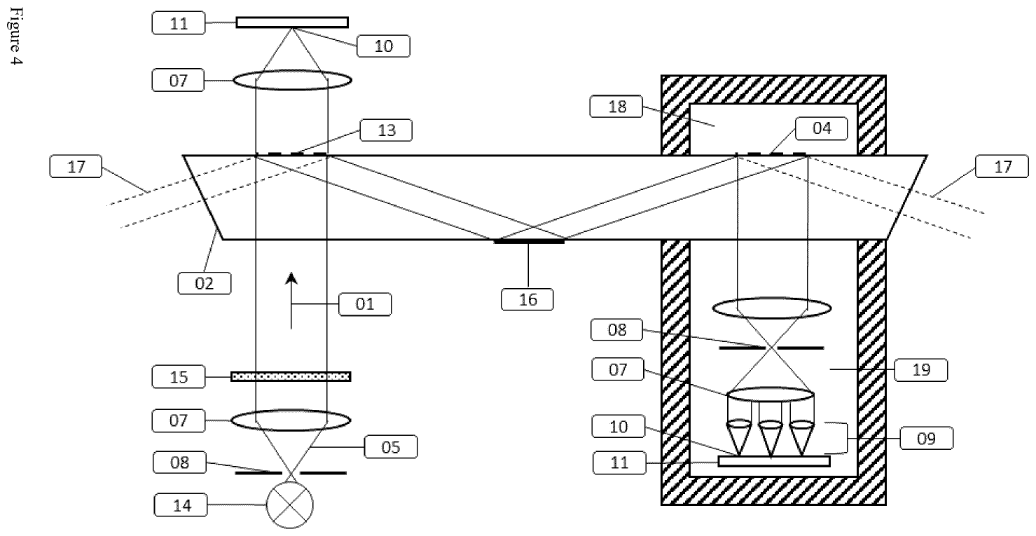

In one embodiment, the grating (04) and binding zone are positioned in a row (single-side replication, FIG. 4). Here, light (01) from light source (14) might be is filtered (15) to become sufficiently monochromatic light or light with a well-defined bandwidth. The light (01) is diffracted by a grating (13), creating a dispersed first or higher order diffracted beam. A portion of the light (01) preferentially the minus first order or transmitted or reflected zero order might serves as internal reference. The major portion of light (01) is reflected by a the rear surface (16) of the sensor chip and is guided to the binding zones (04) inside the flow cell (18) and creates the signal light in the detection space (19) subsequently. Excess light (dotted lines) is led out of the system into beam dumps (17). Alternatively to the setup depicted in FIG. 4, binding zones (04) can be designed in different shapes, as shown in FIGS. 1-3. The beam path can be modulated accordingly.

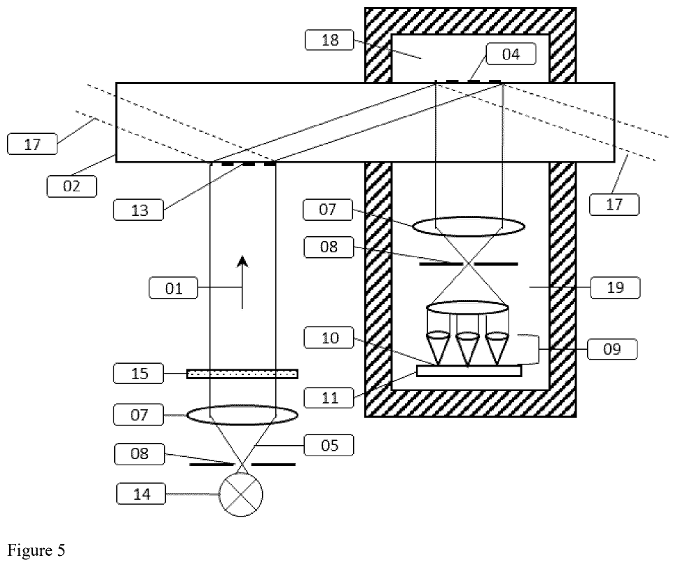

A simpler embodiment of the device illustrated in FIG. 4 is depicted in FIG. 5. Here, the light (01) is diffracted by a grating (13) and directed directly to the binding zone (04) inside the flow cell (18). In this setup, grating (13) and binding zones (04) are positioned on the sensor chip (02) on opposing sides. Alternatively to the setup depicted in FIG. 5, binding zones (04) can be designed in different shapes, as shown in FIGS. 1-3. The beam path can be modulated accordingly.

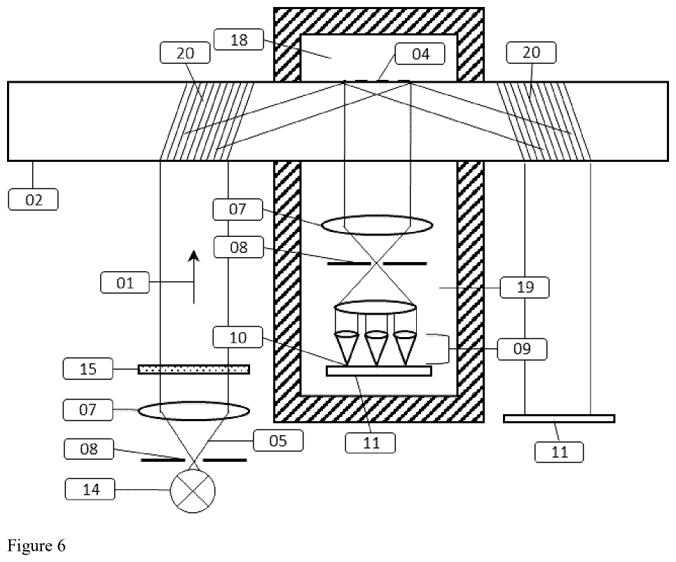

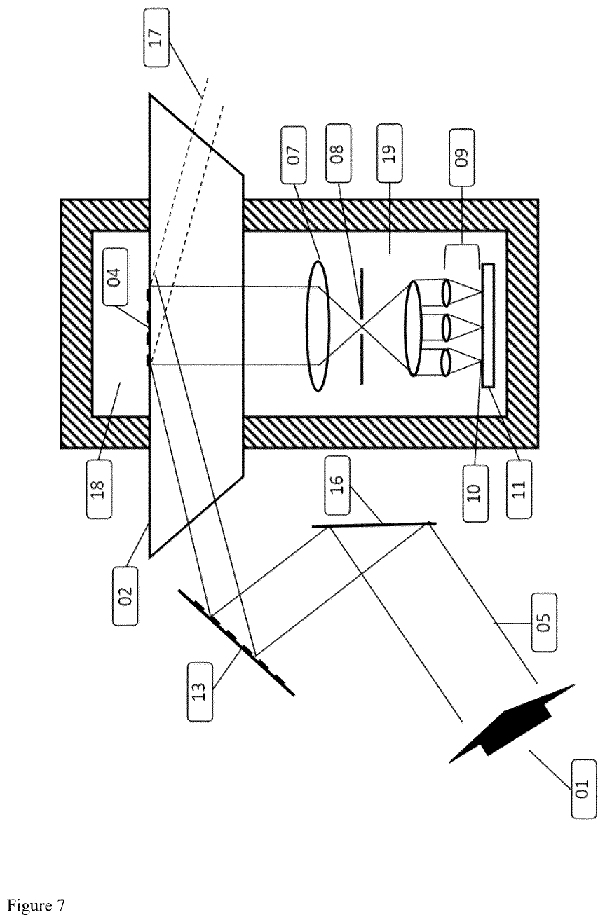

A more advanced version of the previous embodiments (described in FIG. 4 and FIG. 5) contains a 3D volume holographic grating (20) for shaping the excitation light (FIG. 6). The light (01) is guided into a volume hologram (20), creating dispersion and redirecting it to the binding zones (04) inside the flow cell (18). Focal spots (10) are created in a light-covered detection space (19) subsequently. A second volume grating (20) assures scattering and can be used as reference. This setup may result in high efficiency, since a better support of only selected diffraction orders is possible with volume-holographic gratings. The volume-holographic grating may include additional functions, such as collimating and filtering the excitation light. Then, part of the excitation optics (08, 07, 15) could be eventually omitted. In another embodiment, the chip containing the binding zones does not include gratings (FIG. 7).

In another embodiment, the diffractive sensor is adapted for the use of both, coherent and non-coherent light, by installation of a grating outside the sensor chip (FIG. 7). The light (01, coherent or non-coherent) is guided onto a grating outside of the chip (02), creating dispersion and reflecting it to the binding zones (04) at the interface between the chip and the flow cell (18). The dispersion of the light (01) at the external grating (13) is adjusted to compensate the dispersion added at the interface to the chip (02) and the dispersion added by the binding zones (04) itself resulting in a signal beam, where all wavelength from the light source propagate along one axis. The lens (08) has to be sufficiently achromatic to not introduce further chromatic errors. Same is true for the microlens arrays (09). The microlens array may be also replaced by or replaceable within the device by other types of microlens arrays one or two dimensionally arranged or even by just a single lens (not depicted). By this means the system can be adopted to different arrangements of binding zones.

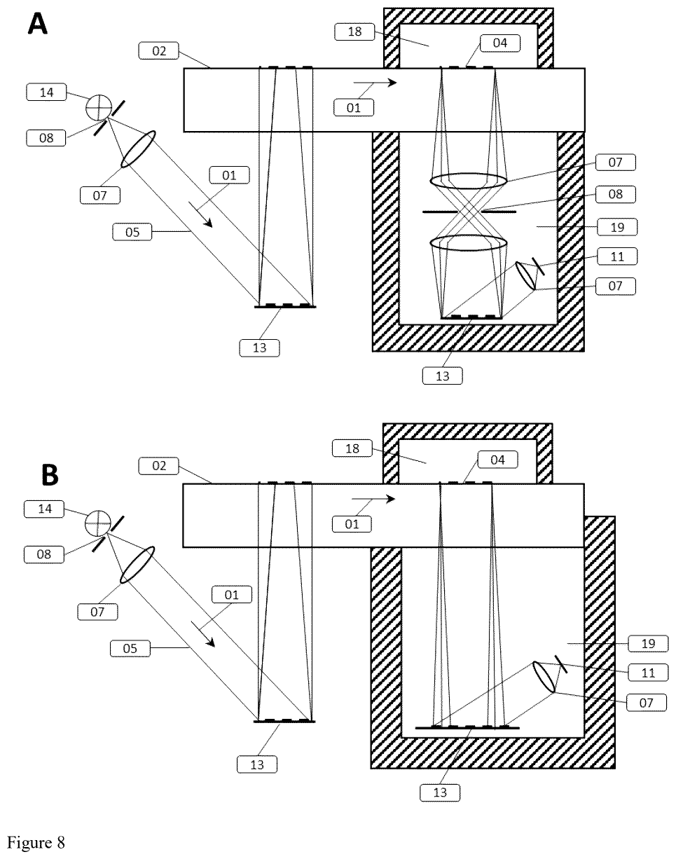

In a further embodiment, grating and binding zones are positioned in row on the sensor chip with waveguide (02) and the light (01) is directed from a first grating (13) to the sensor chip and coupled to the planar waveguide (black arrow) to the binding zones (04, FIG. 8). The dispersion of the first grating is done in a manner that the angle of coupling is optimized for all wavelengths of the light. The first grating may be imaged to the coupling grating of the waveguide (not shown in the Figure). In one version (FIG. 8A), diffracted light from the binding zones (04) is imaged to a second grating by two lenses or objectives (07) forming a focal point between the lenses. An adapted eventually elongated pinhole (08) might be used as a spatial filter to remove stray light. The plane of the pinhole shows a dispersed focus. The light (01) is finally diffracted at a second grating (13) positioned in a conjugated plane to the binding zones, guiding it to the photodetector (11). Here, all the different methods described in the description of the alternative embodiments might replace lens (07) including aperture arrays and microlens arrays. Alternatively (FIG. 8B), light from binding zones (04) is directly guided to the grating (13) as a simpler approach.

In another embodiment not specifically shown in a figure but like FIG. 8A movement of the pinhole (08) along the direction of dispersion would allow for selection of the detection wavelength (not illustrated in the figure). A similar configuration could be also realized in the excitation beam path if the first grating (13) is imaged to the coupling grating forming an intermediate dispersed focus of the exciting light (01). A dynamic motion of even oscillation of one or both pinholes (08)—one in excitation and one in detection—supports a wavelength modulated readout of the sensor chip (02). The motion might be synchronous or asynchronous. In case an asynchronous motion of the pinholes (08) would be applied, e.g., at different frequencies generates a modulation of the signal with mixed frequencies, since the setup acts as two independent monochromators, where the efficiency of the second monochromator is given by the binding of molecules at the binding zones (04). This modulation might help to further suppress straylight especially speckles on the camera or detector. The camera or detector readout must be synchronized or at least sample the modulation generated by the motion of one or both pinholes. In a similar approach modulated wavelength or even only some discrete or only two wavelengths might be applied. The resulting modulation of the signal at the detector can be used to suppress background. In a very simple version of the system, the detector might be replaced by a position sensitive detector or a linear array with two or more elements. The signal may be than extracted from the raw signal by extracting the position modulation.

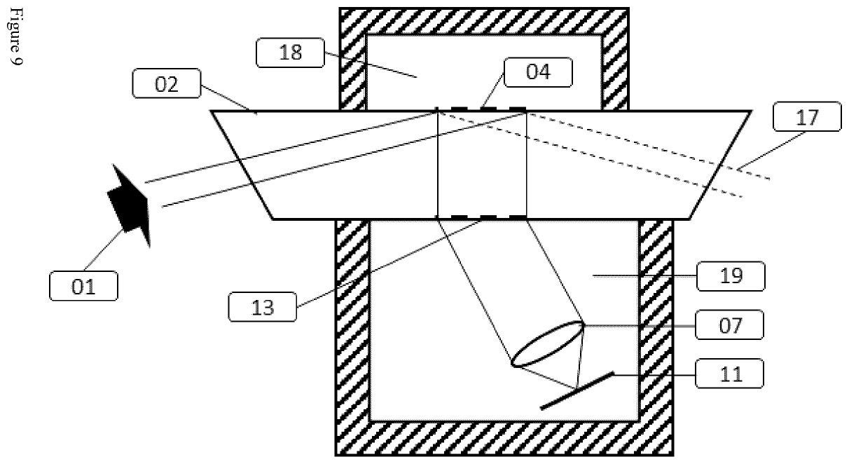

To simplify the optical setup of the device, in one embodiment a grating is positioned downstream of binding zones on the sensor chip (FIG. 9). Light (01) is guided to the binding zones (04) on the upper side of the sensor chip (02). Diffracted light (01) is then directed to a transmission grating (13) on the lower side of the chip (02, parallel to the binding zone 04). The light is there diffracted towards the detection optics (07) that focusses the light on a detector (11). The detection beam path might include other variants shown in the alternative embodiments including multiplexing with microlenses. In a variant of the embodiment not shown in the figures the second grating might be a transmission grating positioned in the instrument in proximity to the chip generating a transmitted beam identical to the one shown in FIG. 9. In a further embodiment of the invention the transmission grating either on the chip or in the instrument may include the focusing function and dispersion function. In this setup, further optics can be omitted.

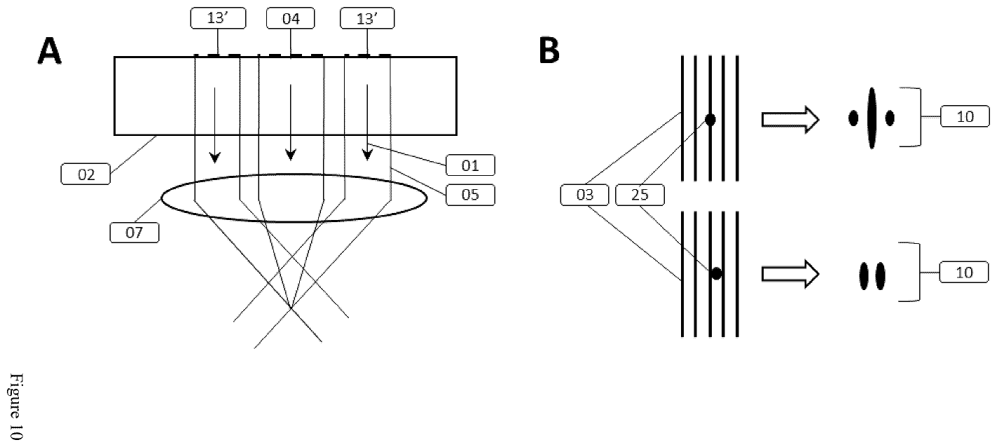

Furthermore, in one embodiment of the of the invention, signal-to-noise ratio of the device can be enhanced on various levels, enabling the signal extraction from the noisy environment: In one case, a spectral-temporal modulation is performed by shifting the spectrum of light send into the beam path of the device in a temporal course (not depicted). For this, several methods would be feasible, as for example switching between different coherent light sources, using a monochromator with modulated transmission wavelength, utilizing a rotating grid or a rotating multilayer filter in the excitation beam path. In another case, a better signal-to-noise ratio can be achieved by modulating the intensity of the excitation light over time (locked-in amplification detection). In a similar manner also the angle of incidence of the excitation light might be modulated. This could be done by galvanometric scanner tilting a mirror, glass plate or grating, acousto-optical deflector, movable slit or diaphragm or other means to change the angle of incidence. The resulting modulation of the signal at the detector is then analyzed. The advantage might be that modulation of the stray-light varies in a different manner than the modulation of the signal contributed by the binding molecules. This allows for a further increase of the sensitivity. In an additional embodiment, the sensor chip contains reference gratings (13′) next to the binding zones (04, FIG. 10).

Here, the light (01) coming from the reference gratings (13′) is shifted in phase and therefor can be used as reference. As the diffracted beams coming out of detection zones and reference gratings are excited with the same incidence wave, a fixed phase relationship is achieved. The different patterns of the focal point (10) projected onto the photodetector (11) provides a specific intrinsic signal, which reveals the relative phase of the signal light (01). The knowledge of the phase of the signal light allows the analysis whether the contribution of the signal is predominantly from the ridges versus the groves of the grating. This feature can be used also for control of quality during an experiment (e.g., analyte binding to binding structures) or also during production. Quality control during production is achieved in case of an inline analysis while the photochemical functionalization or specific binding of the analytes to the ridges or backfilling of molecules to the groves of the grating is done.

FIG. 10A shows a schematic setup, B shows an example how the two different signals might differ at the detector where a contribution of the signal is dominated from ridges versus groves (i.e., a phase difference of Pi/2).

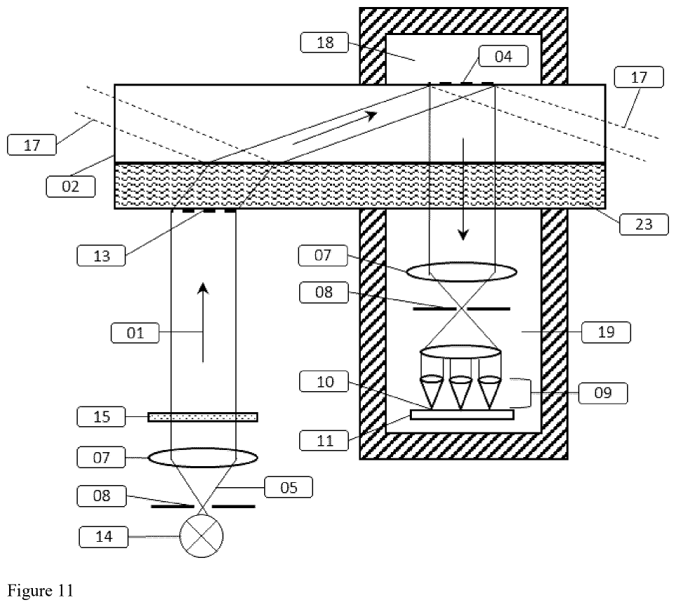

In another embodiment shown in FIG. 11, a grating (13) is optically coupled to the chip (02) with an immersion fluid (23). In case that the refraction index of the fluid is higher than the refraction index in the liquid within the flow cell (18) the grating (13) could be arranged in a simple geometry according to the embodiments shown in FIGS. 4 and 5 where the grating (13) is in a plane parallel to the plane of the binding zone (04). The immersion liquid can be also an adhesive which is supplied with the chip. It can be also supplied with microfluidic channels within the element coupled to the chip. The grating (13) can be holographic, volume holographic or relief grating. The position of the grating can be at the rear or frontside or in the volume of the element coupled to the chip. This element might also contain part of the detection optics (not shown in the figure). The shown embodiment may include other methods of detection as described within the description of the alternative embodiments. Other methods to couple the chip to the instruments might also include prisms with or without generating dispersion at the coupling point of the light.

A different embodiment not shown in the figures defines a method to generate a binder modulation on a sensor chip on binding zone. The method of the state of the art to generate a modulated grating like structure is photochemistry. There typically UV light at a wavelength in the range between 300 and 450 nm is used. The readout of the sensor chips is then performed typically at red or infrared light (>600 nm). This is done to reduce autofluorescence in the optical path and from the sample excited by the evanescent field. It might be advantageous to generate the modulated grating with a similar or identical wavelength than the detection wavelength. The advantage would be that the identical beam path could be utilized for the production of the detection zones that for the analysis. The invention includes a setup where a laser beam is split to two beams where one is send along the excitation beam path and the other in backward direction along the detection beam path. The two beams then interfere at the binding zone generating an interference which can be used to generate the binding zones if the laser has a similar or identical wavelength as the detection light source used for the read out of the sensor. The advantage of the approach is that optical errors in the excitation and detection beam path and also in the sensor is intrinsically eliminated by this method. Photochemistry can be excited if two or more low energy photons combine in a multiphoton excitation process. This is possible if the electric field is high enough. Femto- and picosecond lasers at low repetition rate are able to provide sufficient electric fields even at larger areas. Short laser pulses require a finite spectral bandwidth of the laser. Some of the described embodiments describe versions which eliminate the need to use coherent light for the readout of the sensor. The setup utilizing gratings generate pulse stretching of short pulse lasers. A proper setup of the parameters of the laser including pulse length, repetition rate and power is necessary. It is also of advantage to subsequent illuminate of individual binding zones and thus limit the addressed area. Here, a scanner might be utilized. Methods of temporal focusing can help to confine the multiphoton process to the surface of the chip.

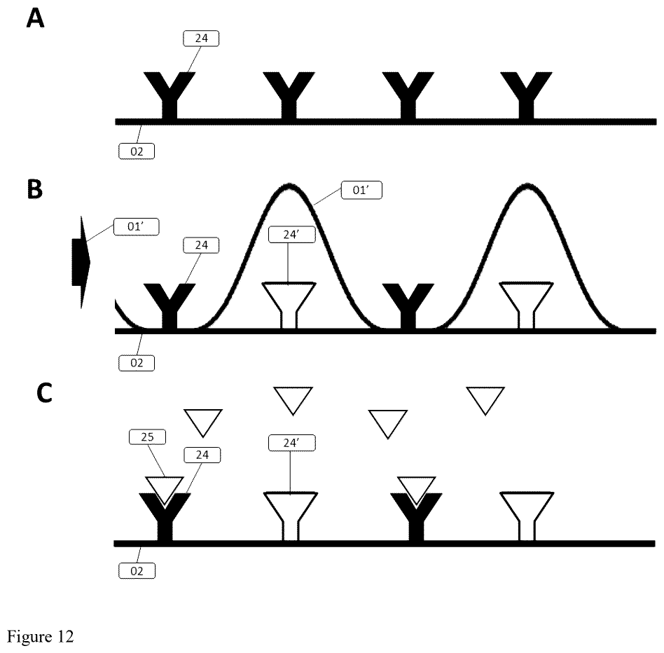

Another embodiment describes a method to modify the sensor chip by using multiphoton excitation at the surface of the chip where two pulsed beams interfere, as illustrated in FIG. 12. Here, binders (24) coupled to the surface of the sensor chip (02) can be partially activated or deactivated (24′) by coherent light (01′) from a pulsed laser (1 Hz-1 MHz repetition, fs- or ps-laser), using the multiphoton effect. The multiphoton effect can be used for denaturation of proteins, causing a modulated analyte affinity without changing the mass density on the surface. Thus, no or little signal offset would be generated by just denaturation of molecules.

To compensate for the spectral bandwidth a grating might be inserted upstream the binding zone and is used to create dispersion of the laser pulse. A wavelength of the pulsed light can be e.g. between 650 to 1000 nm. The photochemical effect is at least proportional to the square of the intensity generating a grating with higher order components. In this example, un-pulsed light at the similar wavelength 650-1000 nm from another e.g., continuous light source can be used for the detection of analytes binding in a diffractive sensor, as described e.g. in FIG. 4. This approach has the advantage that errors occurring during the chip preparation or due to an uneven chip surface are automatically corrected in the detection step. Thus, it even allows to print diffraction structures as an arbitrary speckle pattern with binding sites onto the chip surface instead of a line pattern with binding sites (03) as depicted e.g., in FIG. 1.

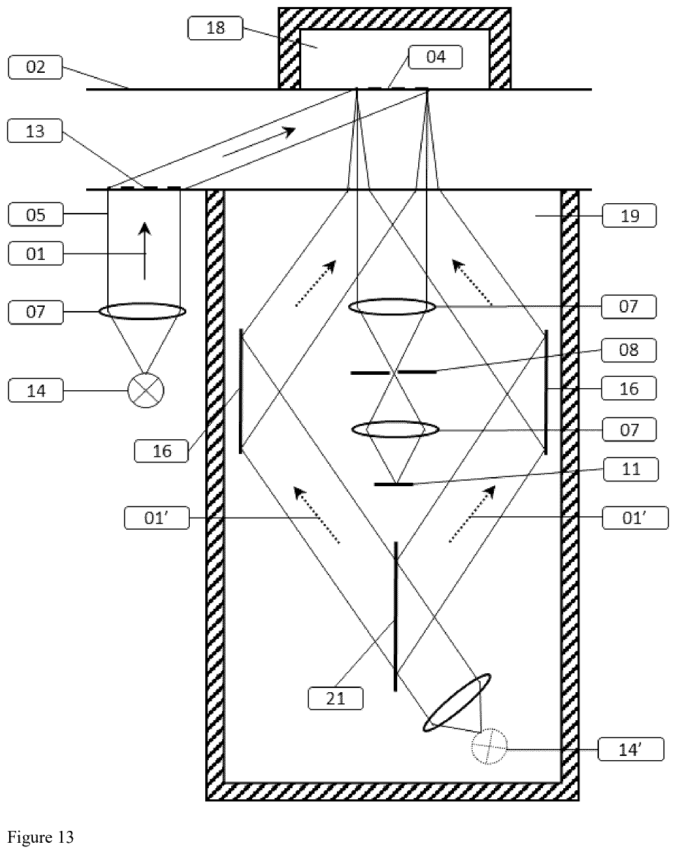

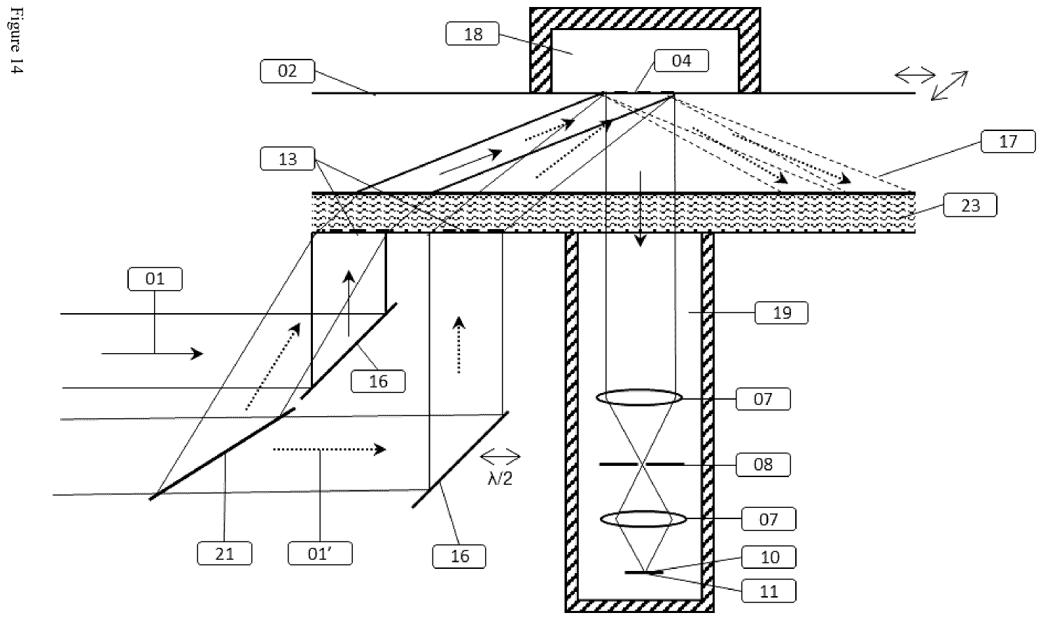

In other embodiments shown in FIG. 13 to FIG. 15, the production of the chip and the analysis is done synchronously. This can be done if the light activating the photochemistry and the detection performed in a pulsed alternating manner, e.g., by switching between the modes at e.g., 0.1 to 100 Hz. It is also possible to use bandwidth filters to suppress the activation light and autofluorescence from the chip and sample. The attachment of the binders or adapter molecules could then be directly monitored. Implementing a phase-shifter (e.g. piczo on a mirror (16 in FIG. 14), tilting plate (21 or 16)), SLM=Spatial Light Modulator) in one of the two excitation beam paths allows a switching of the activations from the bright areas (constructive interference lines) to the dark areas (destructive lines). Thus by exchanging the binders in the liquid from specific ones to unspecific ones or between different adapter molecules in combination with shifting the phase and a readout of the chip allows to generate a well characterized chip with maximum sensitivity. This is true not only for the case of multiphoton excitation but can also be utilized in single photon UV photochemistry. Then, there are different beam path needed for the beam activating the photochemistry and the light for the detection. A device which is able to perform the immobilization of binders and the readout can be used by customers designing their own experimental setup. FIG. 14 shows that the coherent photoactivation light source (01′) is split into two beams of identical intensity. Both are directed towards the chip (02) and interfere at the surface. This directly generates the binding zone (04) necessary to bind the analytes. By shifting the beam splitter (21) or tilting one of the directing mirrors (16) the phase of the interference at the sample can be changed. By this means different molecules can be bound with different modulation phases. It also allows first binding of the analyte with a first modulation phase and a second ‘backfilling’ molecule at a modulation phases shifted with pi/2. The reaction time or intensity can be chosen so that the overall coverage with scattering molecules (analytes and backfilling molecules) is finally homogenous. An in situ control of the diffracted light during the subsequent binding may help to tune the homogeneity of the activated chip and thus to minimize the initial diffraction.

It is of advantage to combine the functionalization of the chip with a direct readout of the immobilized molecules. To achieve this it might be useful to bind a small amount of fluorescent dye molecules with the same grating structure. After washing the additional application of UV or pulsed IR light generates a fluorescence from the dye. The intensity of the fluorescence can then be detected with respect to the phase between the interference structure and the grating. This allows to shift phase so that the intensity is minimized. This process allows a repositioning of the substrate for the sensor chip and still do a proper backfilling of the grit. Thus large areas can be produced at the same time.

In another embodiment of the invention not shown in a figure the angle of incidence of the two light beams which interfere at the surface can be changed and selected. To achieve this a scanner or SLM may be used in a pupil of the illumination optics. This pupil is than imaged with appropriate lenses over the split beam path to the surface of the chip generating gratings with variable grating modulation. This allows for generation of overlaying multiple grating structures subsequently or in the case of the use of an SLM also simultaneously. The use of an SLM or DLP (digital light processor, a MEMS device) also allows a generation of spatially separated sets of gratings arranged e.g. in an array. This makes the production of different kinds of chips versatile and easy. When an SLM or DLP is used different diffraction orders may be used to directly generate the two beams necessary to generate the interference at the sample surface. The use of overlapping gratings in multiplexed assays helps to reduce mass effects, since a larger surface is used for detection. This reduces the need of diffusion or flow in the chip.

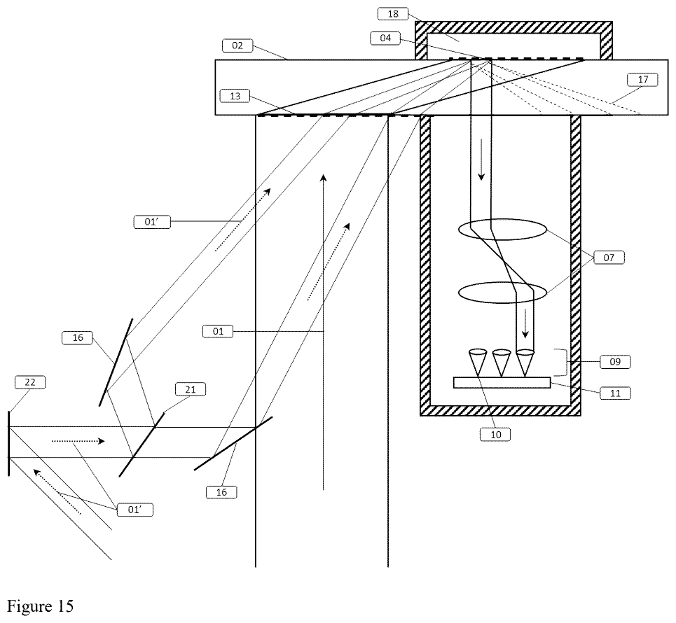

FIG. 15 shows an embodiment where the light beam (01′) for the activation of the chip is modulated by means of an DLP device (22). The modulated beam is than split in two beams (21) and is than redirected to the chip (02). In this example a coupling grating (13) on the chip is used to couple the two beams into the chip and direct them to the surface where the grating is activated, forming a binding zone (04). In this example the same grating can also be used to couple the detection beam (01) into the chip.

FIG. 15 shows the process which is part of the invention to simplify the activation of the chip. The different steps are shown in FIG. 15A:
a Binding zone area of the chip covered with photoprotective groups
b Illumination of distinct areas of the binding zones with UV light to cleave photoprotective groups and attach binders
Measure diffraction signal increase with detection light until binder coverage is sufficient.
d Phase shift and UV illumination of remaining areas to cleave photoprotective groups and attach non-binders
e Measure diffraction signal decrease with red light until binder signal is completely extinct.
f Optional: Application of random phase UV illumination to block remaining binding sites.
g Addition of an analyte to determine concentration and binding kinetics by illumination with red light.

The inserted graph FIG. 15B shows schematically the signal measured during the process. The advantage is, that a precise setting of the final signal can be achieved when the analysis step (c) and (e) is included. The change of signal intensity ( ) over time is modulated by phase shift pi/2 and application of random phase.

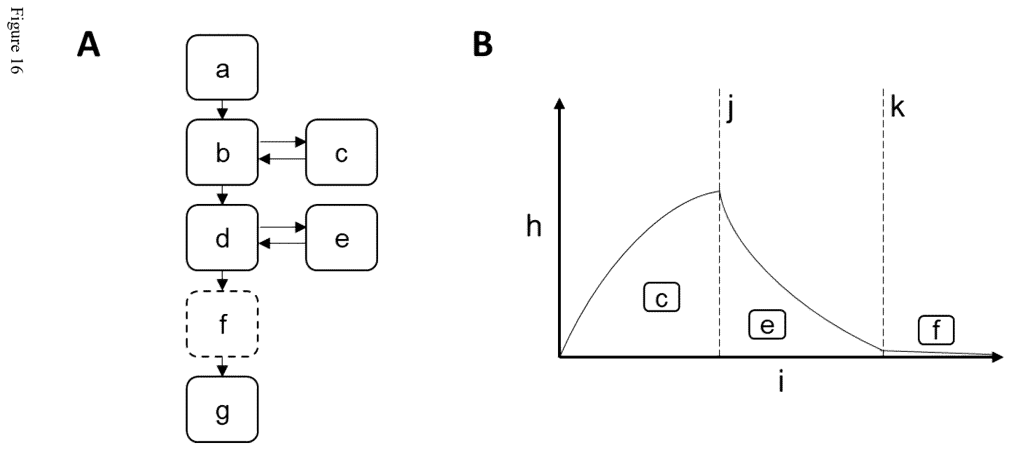

In another embodiment shown in FIG. 16, a method is implemented that allows a strong reduction of speckles occurring from scattering from unspecific background or also from the optical system itself this also includes the case where a single wavelength light source with long coherence length is used. The invention includes a spatio-temporal modulation (26 in FIG. 16A,C) of the light (01) illuminating the surface of the chip or spatio-temporal filtering (26 in FIG. 16B) of the light emitted from the surface of the chip (02) and collected at the detector (11). The filtering might also be a spatio-temporal change in polarization and or introduction of phase-shifts.

Background of the invention is the fact that the overall signal detected at the detector contains light from the molecules of interest arranged in the grid like structure (the ‘real signal’) and light from nonspecific bound molecules arranged statistically on the surface and straylight within the instrument (background signal). Both parts of the signal can be regarded as a sum over the light from all contributing molecules, whereas the ‘real signal’ adds with a well-defined phase and all other contributions add with a statistical distributed phase which generates speckles. These speckles change if the illumination amplitude distribution or polarization distribution with respect to position on the surface changes.

In an embodiment of the invention the ‘real signal’ interferes at the detector (11) coherently at one focus point. This is achieved by focusing the light scattered from the surface with the focusing lens (07). The pupil is arranged at a position with respect to the surface that a diffraction order (typically the first order of diffraction) of the light scattered from the grid like structure is collected by the pupil. The spatio-temporal modulation now introduces in a simple embodiment a spatial varying intensity modulation which effects the distribution of light at the area of the grid like structure on the chip surface (02). In the easiest embodiment a transmission structure ((26) in FIG. 16A,C) is inserted in the illumination beam path and varied. This can be done just by rotating a structured mask in the excitation beam path. The result is, that the signal light which is accumulated at a given time contains contributions of a subpopulation of the molecules of interest and also a subpopulation of the molecules contributing to the background signal and also contributions of the light reflected and scattered within the apparatus. All these contributions change over time in case a spatio-temporal modulation is applied but still maintain the focus of the focus on the detector. In the case the average transmission of the mask would be e.g. 30% the overall signal at the detector would be reduced to an average of 30%. The ‘real signal’ is still a sum over contributions with well-defined phases and would thus be in a first order consideration independent of the position of the mask. The background signal is also reduced to 30% but adds up to a speckle distribution which is changing with the position of the mask. This is also the case for the contribution of the light reflected and scattered within the apparatus. If the detector is temporarily integrating over all or a large enough entity of the time (i.e. positions of the mask) the speckle distribution will average to a spatially smooth signal at the detector. The integration can be either done directly on the detector by accumulating light for a long enough time or by adding several readouts of the detector. In case several readouts are taken correction on the individual data may be undertaken, do further increase the sensitivity. Among those might be shifting the data to correct for aberrations, means to deconvolve the introduced artefacts generated by the modulation, normalization to the actual overall excitation and transmission. The final integration of the speckle-reduced signal can be performed over a moving interval to increase the temporal resolution. The interval might be synchronized to an overall modulation period of the mean used to spatio-temporal modulate the light. The background signal at the position where the ‘real signal’ occurs can be than estimated by an average of the signal over areas where only speckles are expected. The uncertainty of this estimation is much lower for the integrated signal. This holds especially in case that a substantial contribution of the signal originates from reflected and scattered light within the apparatus. This makes the ‘real signal’ distinguishable in space with respect to the background also in cases where the absolute signal of the background exceeds the ‘real signal’. Without the temporal integration the uncertainty of the determination of the background equals the modulation depth of the speckles, which is in the order of the speckle intensity itself. The sensitivity of the system would be limited by shot noise in case a complete constant background is achieved by introducing the spatio-temporal modulation and temporal integration.

In a more precise description the spatial and temporal modulation M(x,y,t) might be a complex vectorial (for the two polarization directions) function multiplied to the field vector components of the wave S(x,y) in the pupil of the detector (or at the surface of the chip). The detector than detects the intensity derived from the Fourier transform of the modulated wave in the pupil which can be calculated as a convolution of the Fourier transform of the M with the Fourier transform of S for each vector component. The time dependent signal at the detector is thus smoothed out and spatially and temporarily modulated. It is thus important, that the Fourier transform of M contains enough low frequency components to keep the final signal still confined on the detector. The overall structure of the mask has to be large with respect to the pitch of the lines in the grating it is ideally in the order of the overall size of the grid structure itself and should not contain Fourier-components close to the Fourier-component of the grit structure. Thus simple macroscopic multiple holes, macroscopic gratings ideally approximately perpendicular to the grit and macroscopic random structures are suitable for the de-speckling of the background. The mask could also contain phase structures if a fraction of the molecules would be still illuminated an undisturbed phase. The mask may be in the excitation beam path close to or also apart from a position conjugated to the grid structure. It can be also in the detection beam path between the chip and the focusing lens or also after the focusing lens.

The generation of the mask may be also done by means of an SLM of DLP device. A SLM might be also used to focus different subareas of the chip related to fractions of the aperture of the SLM to different areas of the detector and modulating the phase of the signal in the fractions of the SLM individually. This can be used to configure the system for different multiplexed applications and maintain the ability to de-speckle the background signal. A DLP may be sued in a similar way. The spatio-temporal modulation might be also just an introduction of a spatial distributed and modulated rotation of the polarization by either 0 or 90°. At a given fraction of the area of approximately 50% of the area the signal interference at the detector is split into two independent subsets generating two independently speckled background signals and ‘real signals’. This already reduces the overall modulation depth of the combined speckle background. The full amount of light is uses and further speckle reduction is achieved by modulating the subarea where the polarization is rotated. A polarization rotation might be introduced by an SLM like using liquid crystals of other masks introducing polarization rotation.

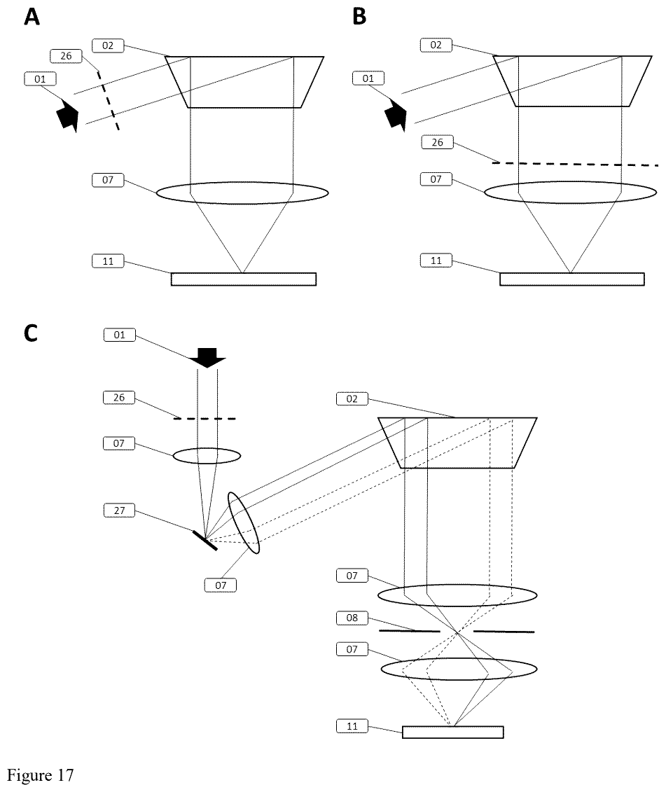

In a further embodiment the layout of the system shown in FIG. 17A,B,C is done in a manner that the mask can also select subareas of the grating to be illuminated. These areas can be assigned to individual binding zones of molecules in a multiplexed setup. The mask may be than still modulating the light within the subarea according to the method described above. The selection of the subarea can then be used to subsequently read the signals from several subareas. One of the subareas may be also used as reference source to determine the absolute phase. This may be illuminated in addition to check for the absolute phase of the signal. One embodiment shown in FIG. 16C might use a scanner or MEMS-scanner (27) to direct the light to the subareas. In the embodiment shown the light scattered from the chip is focused to an intermediate focus in a diaphragm (08) which additionally reduces straylight from the apparatus. The ‘real signal’ is focused down to the same point in the diaphragm which allows for a rather tight filtering of scattered light. The open area of the diaphragm has to be large enough to transmit a sufficient area of speckles from the unspecific background so that a measurement of the background in the vicinity to the focus of the ‘real signal’ is possible on the detector. This mode of subsequent detection of different areas for multiplexed analysis allows high intensities on the areas for short times avoiding contributions of scattered light from other areas of the chip. It allows also to read-out just a tiny part of the sensor or make use of just little sensor. This allows for a fast readout. As an example: If a readout of just 100×100 pixel on a sensor is performed framerates of 10 to 100 kHs is possible. Thus a large multiplexing and digital integration over spatio-temporal modulations for each subarea is possible. A MEMS scanner can be used to modulate the light on the chip. MEMS scanner might be used to illuminate the chip on a subarea moved in a one dimensional manor or in two directions as e.g. a Lissajours figure.

In another embodiment shown in FIG. 17 the spatio-temporal modulation is combined with a chip where the light is coupled to a waveguide. The evanescent field of the light in the vicinity of the surface of the waveguide is than scattered by the grid like molecular structure. In the example shown a scanner is used to address different positions of the coupling grating which than results in a spatio-temporal modulation of the light traveling through the waveguide. This results in a modulation of the speckle background on the detector and could also be used to address different stripes on the chip separately. If the shown embodiment is combined with dispersive elements either in front of the scanner (27) or after the scanner and in the scattered light in front of the lens (07) the setup might be adapted to incoherent broad bandwidth light still maintaining a single signal spot on the camera with a further reduced speckle background.

In another embodiment not shown in a figure but similar to the embodiments shown in FIG. 17B and FIG. 17 a sensor chip with a planar waveguide is used but without a spatio-temporal modulation of the light coupled to the chip but with a spatio-temporal modulation of the scattered light emitted from the chip. The modulator (26) might there be a moving mask a spatial light modulator (SLM) or a DLP. The SLM might be uses just to introduce the spatio-temporal modulation in terms of amplitude and phase (if combined with a polarization analyzer) or polarization direction or also as a selector for different subareas of the chip. This might be also introduced by an DLP in the position (26). An SLM might be also used to introduce a subarea dependent diffraction to generate different signal foci on the sensor for different subareas.

The method to reduce the speckles on the detector might be combines with all other aspects describes in the different embodiments of the invention and added in various means as a person skilled in the art can easily recognize. It is also possible to introduce an angular modulation of the incident light in the case of a chip based on total internal reflection. This Modulation of the Angle results in a modulation of the position of the ‘real signal’ on the detector. In a simple embodiment not shown in a figure this modulation is readout with just a dual pad photodiode or position sensitive detector. A similar result would be obtained by inserting an oscillating mirror in the detection path. This could also be applied for chips with planar waveguide. The modulation of the difference or ratio of the two signals would be dominated by the ‘real signal’ especially in cased where the de-speckling of the background in the vicinity of the real signal is done at a higher frequency than the modulation of the angle of incidence. This is a method to get a very simple and sensitive device for point of care diagnostic or environmental or food analysis.

There are two contributions to the speckles at the detector. A: scattering of light from unspecific bound statistically distributed molecules and B: scattering from surfaces of the apparatus and from the sensor chip. In a first step the contribution A is considered in a simplified presentation. The number of unspecific bound molecules shell be ‘n’. Each individual molecule contributes with a probability p=0.5 constructively to any given spatial frequency transmitted through the optical system and with q=1-p=0.5 destructively. This is also true for the spatial frequency of the grid The expectation value of the electrical field at this spatial frequency is thus 0.5 n and thus the expectation value sum of the electrical field over the constructive and destructive subset of molecules is zero. The electrical field is gaussian distributed for sufficiently large number of molecules n (with a variance sigma\hat{ }2=npq=0.25 n. The Intensity is proportional to the square of the electric field. Therefore the speckle amplitude scales with sqrt(npq). In the given case that ‘m’ would be the number of specifically bound molecules the contribution would be proportional to m since all electric field vectors add coherently. The overall detection limit thus limited at m>˜sqrt(npq)=0.5 sqrt(n) This reflects the statistical contribution of molecules arbitrarily bound to the spatial frequency selected for the specific binding of the target molecules. In one embodiment of the invention not shown in a figure the detection limit is increased by changing the factor p towards larger or smaller values. As an example if p=0.01 the detection limit would be m>˜sqrt(n*0.01*0.99)=˜0.099 sqrt(n) which is already an increase of a factor of 5 in sensitivity. Reducing of increasing the probability p is achieved by narrowing of widening of the lines of the grid. This could be done by several means. Dedicated masks, multiphoton supported immobilization, saturation could be used. In case activation is saturated with an illumination pattern generated by an interference of two planar waves for a long time only small lines would not be covered in the vicinity of the lines where the electrical field of the interference is zero. This consideration holds only if the stronger confinement of the structure is taken into account in the readout system. This requires to read all added spatial frequencies i.e. additionally not only the first but a series of higher diffraction orders. These could be collected as a series of foci on the detector. Each focus represents one order of diffraction and has its own independent speckle background. The signal of all detected diffraction orders has to be accumulated to achieve the signal with increased sensitivity.

In another embodiment not shown in a figure a different mean of speckle reduction is introduced. This can be combined with most of the embodiments described in this disclosure and addresses the light scattered within the apparatus In case that the incoming and outcoming beam light used to excite and detect the signal is approximately collimated a displacement of the chip does only effect absolute phases between incoming and outcoming beams but not relative phases within the scattered beam. Thus a displacement of the chip introduces phase differences between scattered with origin from the excitation beam path and scattered light with origin from the apparatus after emitted from the chip since the overall beam pathlength of the scattered light changes due to the displacement of the chip with respect to the apparatus. A random or vibrational displacement introduced for example by an piezo-transducer thus eliminates speckles from scattered light with origin from the apparatus. A pure displacement without any tilt of the chip preserves the position of the focus of the diffraction orders on the detector and thus increases the signal-to-noise ratio significantly in cases of a dominant contribution of light scattered from the apparatus.

1. Device for the detection of target molecules, comprising
a transparent substrate provided with binding sites on one surface of the substrate, wherein the binding sites are capable of binding at least one target molecule at least one light source
means for coupling light provided by the light source into the substrate, wherein at least a part of the light generates an evanescent field of light propagating along the surface provided with the binding sites,
wherein the evanescent field of light is diffracted by target molecules bound to the binding sites, thereby creating at least one detection signal which is detected by at least one detector
characterized in that the light source provides low coherent or non-coherent light and the dispersion of the detection signal generated by the diffraction of low coherent or non-coherent light is reduced by at least 50% by one or more dispersive elements.

2. Device according to claim 1 characterized in that the spectral bandwidth of the light is wider than 1/100, typically wider than 1/1000 or wider than 1/10000 of the center wavelength of the light.

3. Device according to claim 1 characterized in that the dispersive elements are located in the path of light before and/or after the light is diffracted by the target molecules bound to the binding sites.

4. Device according to claim 1 characterized in that the dispersive elements are gratings and/or coupling gratings and/or prisms and/or volumetric holograms and/or tilted interfaces.

5. Device according to claim 1 characterized in that a plurality of binding sites is provided at different locations on the substrate, thereby generating a plurality of detection signals and wherein the detection signals are individually focused on the detector using at least one array of optical elements.

6. Device according to claim 5 characterized in that the array of optical elements comprises lenses and/or microlenses and/or facetted elements and/or rotating apertures or sliding apertures.

7. Device according to claim 1 characterized in that different arrays of optical elements with different optical properties are provided wherein the different arrays of optical elements are exchangeable.

8. Device according to claim 1 characterized in that the arrays of optical elements are provided with at least one aperture plate and/or an aperture plate array.

9. Device according to claim 1 characterized in that the detection signals are space-filtered by at least one optical element located in the Fourier plane of the plane of the binding sites.

10. Device according to claim 9 characterized in that the optical elements for space filtering may select at least one diffraction order of the detection signal.

11. Device according to claim 1 characterized in that the substrate comprises a planar waveguide and/or a prism.

12. Device according to claim 1 characterized in that the substrate has at least one coupling surface for means for coupling a beam of light.

13. Device according to claim 1 characterized in that the binding sites are arranged in a plurality of lines having the same or different pitch.

14. Device according to claim 1 characterized in that the light provided by the light source is spatio-temporal modulated with a variable mask, a spatial light modulator or a scanner.
